(19) United States
(12) Patent Application Publication
(10) Pub. No.: US 2015/0080756 A1
(43) Pub. Date: Mar. 19, 2015

(54) ESTIMATING AROUSAL STATES

(71) Applicant: THE UNIVERSITY OF SYDNEY, Sydney, New South Wales (AU)

(72) Inventors: Peter Alexander Robinson, Berowra (AU); Andrew John Kelvin Phillips, Wallarah (AU); Matthew James Barton, Rushcutters Bay (AU); Jong Won Kim, Liberty Grove (AU)

(73) Assignee: THE UNIVERSITY OF SYDNEY, Sydney, New South Wales (AU)

(21) Appl. No.: 14/367,409

(22) PCT Filed: Jan. 17, 2013
(86) PCT No.: PCT/AU2013/000033

§ 371 Date: Jun. 20, 2014

(30) Foreign Application Priority Data
Jan. 27, 2012 (AU) 2012900314

(51) Int. Cl. A61B 5/18; A61B 5/11; A61B 5/01

(52) U.S. Cl. CPC A61B 5/18; A61B 5/01; A61B 5/1118; A61B 5/1104
USPC 600/549; 600/300; 600/595

(57) ABSTRACT

Disclosed is a method of estimating an arousal state of a person, the method comprising: providing one or more measurements of a physiological or behavioural variable relevant to the arousal state of the person; and updating a nonlinear physiologically based time-dependent model of arousal using the one or more measurements of the physiological or behavioural variable, thereby estimating an augmented state vector of the model, wherein the estimated augmented state vector of the model includes the arousal state of the person and one or more parameters of the model.

### FIELD

The present invention relates generally to sleep dynamics in mammals and, in particular, to monitoring, predicting, and influencing states of arousal in humans.

### BACKGROUND

Fatigue is a major problem in the workplace for both performance and safety reasons, especially for workers who must perform high risk and/or precise motor-based or decision-making tasks at all hours of the day, such as machinery operators, pilots, surgeons, and nuclear technicians. Fatigue impairs the reactions of such workers to hazardous situations and compromises general performance. Fatigue in general results from sleep disruption or “circadian misalignment”, that is, a body's environment becoming out of phase with its natural sleep/wake (arousal) cycle as a result of long-distance travel. Fatigue may also result from medical conditions such as sleep apnea.

Proper management of working schedules can help reduce fatigue, but in many occupations some degree of sleep disruption or circadian misalignment is inevitable no matter how carefully schedules are managed. As a result there is a need for systems that are able to monitor fatigue in a given individual and predict when it will reach potentially hazardous levels, and where practical, counteract its effects by the use, or recommended use, of external stimuli. Such systems may also find application in counteracting conditions such as sleep apnea and insomnia. External stimuli that affect the human arousal cycle fall into two categories: those that promote wakefulness, such as stimulant drugs, bright lights, and loud noises, and those that promote sleep, such as sedative drugs, dim lights, and inactivity. To be useful, such systems should be based on a physiologically based, experimentally verified model of the human arousal system that incorporates the brain physiology of arousal and the effects of external stimuli upon it.

Physiologically based mathematical models of the human arousal system that incorporate the effects of external stimuli have been proposed in recent years. The model parameters may be estimated from experimental data, and conversely, the model behaviour under different constraints gives insight into the mechanisms underlying certain observed phenomena. However, the highly nonlinear nature of the models has raised difficulties in estimating arousal state from experimental data. In addition, the calibration of model parameters has been based on averages over populations of measured subjects for such observed phenomena as sleep duration and frequency. The applicability of the models to a given individual, as required in such applications, is therefore limited.

### SUMMARY OF INVENTION

It is an object of the present invention to substantially overcome, or at least ameliorate, one or more disadvantages of existing arrangements.

Disclosed are systems, methods, and devices that can estimate and predict the arousal state of a given person. The disclosed system makes use of a physiologically based mathematical model of the human arousal system comprising a set of equations in multiple physiological variables using a set of tuneable parameters. The system includes one or more sensors that provide measurements of certain physiological and behavioural properties of the person, such as activity, light exposure, and core temperature, that are relevant to the model. The system also includes a computing device that applies state estimation techniques to the measured data to simultaneously estimate the model state variable values and the model parameters. Inputs such as light, noise, and stimulant and sedative drug intakes may be incorporated into the model, and techniques from control theory may be applied to predict model behaviour under future scenarios for such inputs and thereby recommend changes to such inputs to delay or counteract the effects of fatigue.

According to a first aspect of the present invention, there is provided a method of estimating an arousal state of a person, the method comprising: providing one or more measurements of a physiological or behavioural variable relevant to the arousal state of the person; and updating a nonlinear physiologically based time-dependent model of arousal using the one or more measurements of the physiological or behavioural variable, thereby estimating an augmented state vector of the model, wherein the estimated augmented state vector of the model includes the arousal state of the person and one or more parameters of the model.

According to a second aspect of the present invention, there is provided a system for estimating an arousal state of a person, the system comprising: one or more sensors configured to measure one or more measurements of a physiological or behavioural variable relevant to the arousal state of the person; a memory, and a processor configured to execute program instructions stored in the memory, the program instructions being configured to cause the processor to update a nonlinear physiologically based time-dependent model of arousal using the one or more measurements of the physiological or behavioural variable, thereby estimating an augmented state vector of the model, wherein the estimated augmented state vector of the model includes the arousal state of the person and one or more parameters of the model.

According to a third aspect of the present invention, there is provided computer program code configured to cause, when executed by a processor, the processor to carry out a method of estimating an arousal state of a person, the method comprising updating a nonlinear physiologically based time-dependent model of arousal using one or more measurements of a physiological or behavioural variable relevant to the arousal state of the person, thereby estimating an augmented state vector of the model, wherein the estimated augmented state vector of the model includes the arousal state of the person and one or more parameters of the model.

Other aspects of the invention are also disclosed.

### BRIEF DESCRIPTION OF DRAWINGS

At least one embodiment of the present invention will now be described with reference to the drawings, in which:

FIG. 1A is a schematic representation of the elements of a physiologically based mathematical model of the human arousal system, and their interactions;

FIG. 1B is a block diagram of the model oscillator used in a variant of the Phillips-Robinson (PR) model;

FIG. 2 illustrates the sigmoid function for typical values of parameters and arguments;

FIG. 3 contains plots of the homeostatic sleep drive, the circadian sleep drive, and the total sleep drive for a sleep-wake cycle simulated using the model of FIG. 1A;

FIG. 4 contains plots of simulated time series for the MA potential and the VLPO potential for the simulated sleep-wake cycle of FIG. 3;

FIG. 5 contains a plot of the MA potential against the total sleep drive for the simulated sleep-wake cycle of FIG. 3;

FIG. 6 contains plots of raw and preprocessed actigraphy data over a 72-hour period;

FIG. 7 is a flow diagram of a method of estimating the arousal state and model parameters of a particular person, at each time instant;

FIGS. 8A and 8B collectively form a schematic block diagram of a general purpose computer system on which the method of FIG. 7 may be implemented;

FIGS. 9A and 9B collectively form a schematic block diagram representation of an electronic device upon which the method of FIG. 7 may be implemented; and

FIG. 10 contains plots of the inputs to and outputs of the UKF when run on a measurement data set representing a 72 hour period.

### DESCRIPTION OF EMBODIMENTS

Where reference is made in any one or more of the accompanying drawings to steps and/or features, which have the same reference numerals, those steps and/or features have for the purposes of this description the same function(s) or operation(s), unless the contrary intention appears.

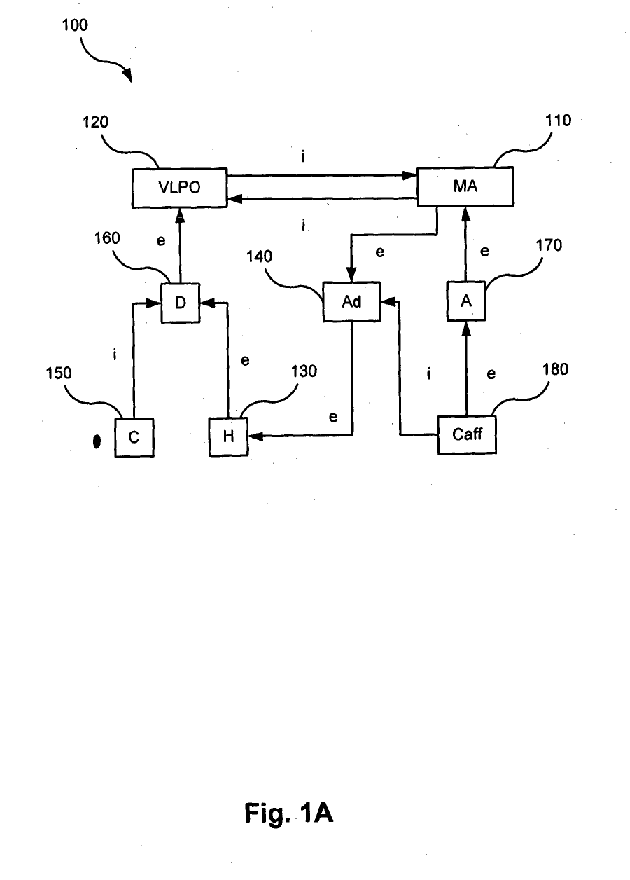

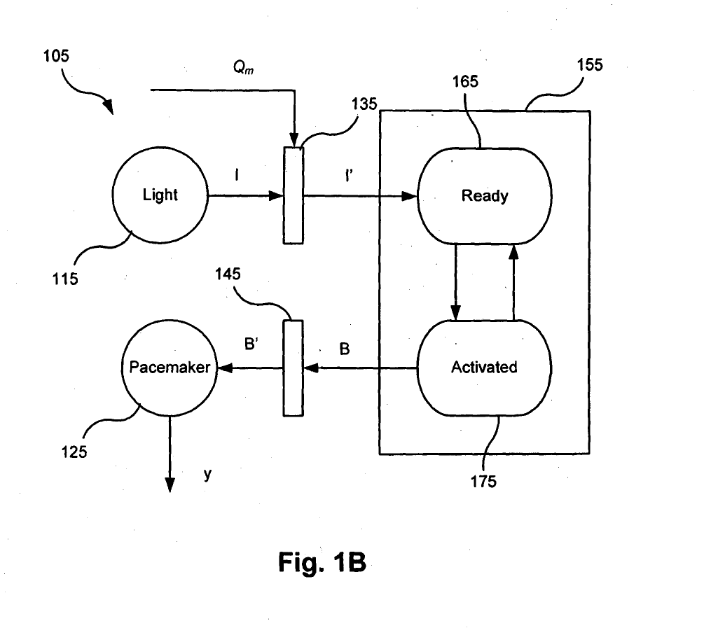

The disclosed systems and methods are based on a physiologically based mathematical model of the human arousal system, referred to herein as the Phillips-Robinson (PR) model. FIG. 1A is a schematic representation 100 of the elements of the model and their interactions.

The overall arousal state of the brain is controlled by the ascending arousal system (AAS), a collection of brainstem and hypothalamic nuclei (neurons) that promote wakefulness. The nuclei of the AAS are mostly either monoaminergic (MA) or cholinergic (ACh). The MA group is represented in FIG. 1A by an element 110 labelled as “MA”. These two AAS groups of nuclei project from the upper brainstem to the hypothalamus and diffusely to the cerebral cortex. The ACh group fires moderately during wake, while it fires with higher and lower rates in REM and non-REM sleep respectively. In the PR model, the ACh group is approximated as having a constant activity level, smoothing out the “ultradian” (within sleep) oscillation while preserving circadian (daily) sleep-wake dynamics. The effect of the ACh group is to excite the MA group via the neurotransmitter acetylcholine, and is represented in FIG. 1A by the element 170 labelled as “A”, with an excitatory connection to the MA element 110. The A element 170 also includes the effects of other neurotransmitters such as Orexin.

The ventrolateral preoptic (VLPO) nucleus of the hypothalamus is regarded as the sleep center of the brain as it fires rapidly during sleep but not in wake. The VLPO acts via the neurotransmitter GABA and has a descending projection to the AAS. The VLPO and MA nuclei are mutually inhibitory; i.e., the firing of one population inhibits the other, and vice versa. The VLPO group of nuclei is represented in FIG. 1A by the VLPO element 120, with mutually inhibitory connections between the MA and VLPO elements 110 and 120.

During wake, sleep-promoting agents called somnogens accumulate in the basal forebrain, and act to inhibit the populations that inhibit the VLPO, eventually disinhibiting (exciting) the VLPO and promoting sleep. This effect is incorporated in the PR model via the homeostatic sleep drive H, and is represented in FIG. 1A by the element 130 labelled as “H”. Adenosine (Ad) has been proposed to be a somnogen responsible for this homeostatic drive, although other somnogens may be involved, including cytokines which are produced by the immune system during illness. In the PR model, the homeostatic sleep drive H is related to the concentration of somnogens in the basal forebrain, which is represented in FIG. 1A by an element 140 labelled as “Ad”, with an excitatory connection to the H element 130. The production rate of adenosine generally correlates with the activity of the MA group of nuclei as described below, so FIG. 1A shows an excitatory connection between the MA element 110 and the Ad element 140.

The suprachiasmatic nucleus (SCN), which is the circadian pacemaker in mammals, and which is synchronised (entrained) with ambient light, outputs a circadian drive (C) which oscillates with a period of 24 hours and is relayed to the VLPO population, both directly and via the dorsomedial hypothalamus. This circadian drive C is represented in FIG. 1A by an element 150 labelled “C”. The circadian and homeostatic drives combine to produce an overall sleep drive D to the VLPO with 24-hour periodicity, represented in FIG. 1A by an element 160 labelled as “D”, with an excitatory connection to the VLPO element 120.

This periodic drive behaviour and the mutually inhibitory relationship of the VLPO and MA nuclei give rise to a model behaviour analogous to that of a flip-flop circuit in electronics. Such systems exhibit two stable states, with only one element active in each state, and rapid transitions between states. Thus, the stable sleep and wake states are robust, and no stable intermediate states exist. At any time, one population of nuclei dominates (either the MA during wake or the VLPO during sleep), with rapid transitions between them due to changes in the sleep drive D.

In the PR model, each population of nuclei is described by its mean cell body (soma) potential (in volts) as a function of time, denoted by Vⱼ(t), where the subscript j indicates the population (v for the VLPO group, m for the MA group). The mean firing rate (or activity) Qⱼ of a population, in Hz, is modelled by a sigmoid function of the soma potential Vⱼ of the population:

$${Q}_{j} = \frac{{Q}_{max}}{1 + exp [ \frac{- ( {V}_{j} - θ )}{{σ}^{′}} ]} \qquad ( 1 )$$

where Qₘₐₓ is the maximum firing rate, θ is the mean firing rate threshold, and

$$\frac{{σ}^{′} π}{\sqrt{3}}$$

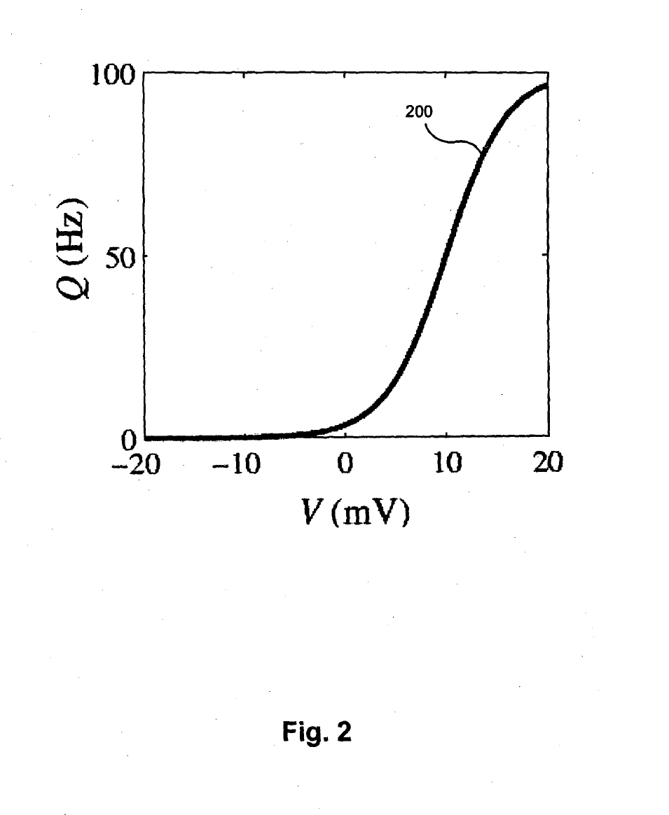

is the standard deviation of the threshold θ, determining the width of the sigmoid. FIG. 2 illustrates the sigmoid function Q(V) 200 for typical values of parameters Qₘₐₓ, θ, and σ′ and arguments V.

The interactions of the VLPO and MA populations are modelled as

$$\begin{matrix}τ \frac{d {V}_{v}}{d t} = - {V}_{v} + {v}_{vm} {Q}_{m} + D \\ and & ( 2 ) \\ τ \frac{d {V}_{m}}{d t} = - {V}_{m} + {v}_{mv} {Q}_{v} + A & ( 3 )\end{matrix}$$

where τ represents the decay time parameter of the neurotransmitters expressed by the VLPO and MA populations (assumed to be equal), vⱼₖ represents the connection strength to population j from population k, D represents the combined circadian and homeostatic sleep drives to the VLPO population, and A represents the excitatory input to the MA population due to acetylcholine produced by the ACh group, and other neurotransmitters.

The overall sleep drive D to the VLPO population is modelled as a linear combination of the circadian and homeostatic sleep drives C and H:

D=v_{vc}C+vᵥₕH (4)

where v_{vc} and vᵥₕ are the connection strengths for the circadian and homeostatic drives, respectively, to the VLPO population. The sign of the parameters vᵥₘ, vₘᵥ, v_{vc} and vᵥₕ indicates whether the corresponding connection is excitatory (positive) or inhibitory (negative).

In one implementation, the circadian drive C is modelled as a sinusoid:

C(t)=c₀+cos [Ωt+φ₀] (5)

where Ω is the angular frequency of the oscillation, φ₀ is the phase of the oscillation at t=0, c₀ is the drive offset, and the amplitude of C is absorbed into the parameter v_{vc}. In a variant of the PR model, described below, a more detailed circadian model is used to model the circadian drive C so as to handle situations where the sleep time changes relative to the daylight hours, such as shiftwork and jet lag.

As mentioned above, the homeostatic drive H is proportional to the concentration of somnogens in the basal forebrain. Levels of somnogens, principally adenosine (Ad), increase during wake due to metabolic activity and decrease during sleep when clearance exceeds production. The clearance rate is modelled as proportional to the concentration of somnogens, while production is modelled as a function of MA activity Qₘ, since MA activity is known to be well correlated with arousal state. The homeostatic drive H is therefore modelled as:

$$χ \frac{d H}{d t} = - H + P ( {Q}_{m} ) \qquad ( 6 )$$

where χ is the characteristic time for somnogen clearance and P is a production function that models the rate of somnogen production in the basal forebrain. During sleep, Qₘ˜0, P(Qₘ)˜0, and somnogens are cleared exponentially at the clearance rate 1/χ. The production function P is modelled as a saturating function of Qₘ:

$$P ( {Q}_{m} ) = \frac{μ}{1 + exp [ \frac{- ( {Q}_{m} - {Q}_{H} )}{δ {Q}_{H}} ]} \qquad ( 7 )$$

where μ is the maximum somnogen concentration and Q_{H} and δQ_{H} are parameters describing the shape of the saturation curve.

The PR model is “input-driven” in the sense that it can incorporate external inputs in the model equations. Examples of such inputs are light exposure, noise, and stimulant and sedative drug intakes. A particular example of incorporating the stimulant caffeine as an input to the PR model is now described.

Caffeine is modelled using a one-compartment approximation, which treats the brain and body as a single well-mixed container. Under this approximation, the concentration Z_{C}(t) of caffeine in the body resulting from a discrete dose of γ (in milligrams per kilogram of bodyweight) at time t₀ is given by

Z_{C}(t)=γ└exp(−kₑ(t−t₀))−exp(−kₐ(t−t₀))┘ (8)

where kₐ and kₑ are the rate constants of absorption and elimination, respectively.

Caffeine is a competitive antagonist of adenosine, binding to the Ad receptors in the brain. The first effect of this mechanism on the PR model is to reduce the coupling constant vᵥₕ between the homeostatic sleep drive H and the VLPO population by an amount proportional to the concentration Z_{C}(t) of caffeine in the body:

vᵥₕ→vᵥₕ[1−ζ_{H}Z_{C}(t)] (9)

where the parameter ζ_{H} is a positive constant representing the masking strength. This first effect is represented in FIG. 1A by an element 180 labeled “Caff” with an inhibitory connection to the Ad element 140.

The second effect of caffeine is an increase in the firing rate of the ACh group of nuclei, modelled by increasing the value of A, the excitatory input to the MA population due to acetylcholine, by an amount proportional to the concentration Z_{C}(t) of caffeine in the body:

A→A[1+ζ_{A}Z_{C}(t)] (10)

where the parameter ζ_{A} is a positive constant of proportionality. This second effect is represented in FIG. 1A by an excitatory connection between the Caff element 180 and the A element 170.

In the variant of the PR model mentioned above, the circadian drive C is modeled using a simplified version of the Jewett-Kronauer oscillator model. FIG. 1B is a block diagram of the model oscillator used in the variant PR model. The oscillator model involves two components: retinal processing of photic stimuli, and a pacemaker process.

The retina is represented by the element 155. Photoreceptors on the retina are converted from the ready population 165 to the activated population 175 by photons at a rate α, dependent on the light intensity I′ reaching the retina. Activated photoreceptors are converted back to ready at a constant rate β. The fraction n of photoreceptors that are in the activated population 175 thus obeys

$$\frac{d n}{d t} = λ [ α ( 1 - n ) - β n ] \qquad ( 11 )$$

where 1/λ is the receptor time constant, and the activation rate α is given by

$$α = {{α}_{0} ( \frac{{I}^{′}}{{I}_{0}} )}^{p} \qquad ( 12 )$$

The photic drive B′ from the retina 155 is modeled as proportional to the rate α(1−n) at which photoreceptors are activated in the retina 155:

B′=Gα(1−n) (13)

where G is a constant. The filter 145 is included to account for the experimentally observed circadian phase dependence of light sensitivity:

B=B′(1−bx)(1−by) (14)

where B is the resultant photic drive to the pacemaker process 125, b is a constant, and x and y are variables of the pacemaker process 125.

The pacemaker process 125 is modeled as follows:

$$\begin{matrix}κ \frac{d x}{d t} = γ ( x - \frac{4 {x}^{3}}{3} ) - y [ {( \frac{24}{f {τ}_{c}} )}^{2} + kB ] & ( 15 ) \\ κ \frac{d y}{d t} = x + B & ( 16 )\end{matrix}$$

where y represents pacemaker activity and x is an auxiliary variable. The parameter τ_{c} is intrinsic period, f fixes the period at τ_{c}, γ is the stiffness of the pacemaker 125, and κ=(12/π) hours.

In the variant PR model, the circadian drive C is modeled as a linear function of the pacemaker activity y:

$$C = \frac{1 + y}{2} \qquad ( 17 )$$

The light intensity I′ reaching the retina is the environmental light I, gated by a gate 135 that depends on arousal state, to model eye closure during sleep. The environmental light I is modeled as a sinusoid on a twenty-four hour period:

$$I ( t ) = \frac{{I}_{A} ( 1 + cos ω t )}{2} \qquad ( 18 )$$

where ω=2π/24, and I_{A}=10⁴ lux, and t=0 corresponds to midday.

The gating function 135 is modeled as

I′(t)=Θ(Qₘ−Qₘ^{th})I(t) (19)

where Θ is a step function, Qₘ is the firing rate of the MA group of nuclei, and Q^{th}ₘ is a threshold firing rate above which the arousal state is defined as wake.

Typical parameter values for the PR model as defined in equations (1) to (10), determined by physiological constraints and comparison with experimental results, are given in Table 1.

TABLE 1
Parameter values for the PR model

| Parameter | Value | Units |
|---|---|---|
| Qₘₐₓ | 100 | s⁻¹ |
| θ | 10 | mV |
| σ′ | 3 | mV |
| τ | 10 | s |
| vᵥₘ | −2.1 | mV s |
| vₘᵥ | −1.8 | mV s |
| A | 1.3 | mV |
| v_{vc} | −2.9 | mV s |
| vᵥₕ | 1.0 | mV s |
| c₀ | 4.5 | — |
| Ω | 2π/24 | h⁻¹ |
| φ₀ | 0 | — |
| χ | 45 | h |
| μ | 4.4 | nM s |
| Q_{H} | 2.5 | s⁻¹ |
| δQ_{H} | 0.1 | s⁻¹ |
| kₐ | 1.0 × 10⁻³ | s⁻¹ |
| kₑ | 4.5 × 10⁻⁵ | s⁻¹ |
| ζ_{A} | 0.15 | mV (mg/kg)⁻¹ |
| ζ_{H} | 0.1 | (mg/kg)⁻¹ |

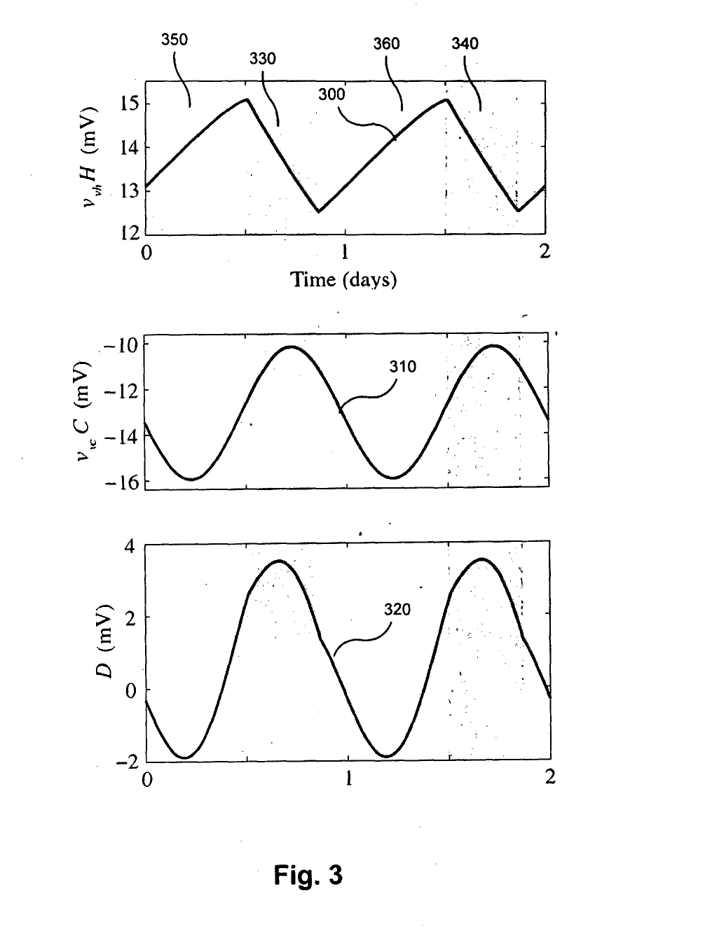

Normal sleep-wake behaviour may be simulated by integrating the PR model of equations (1) to (10) with no caffeine input and the parameter values of Table 1 using conventional numerical integration. FIG. 3 contains plots of the (scaled) homeostatic sleep drive vᵥₕH 300, the (scaled) circadian sleep drive v_{vc}C 310, and the total sleep drive D 320 obtained from such a simulation. The periodic oscillation of the total sleep drive D 320 drives the system back and forth between alternating periods 330, 340 of sleep (shaded) and periods 350, 360 of wake (unshaded), as explained below.

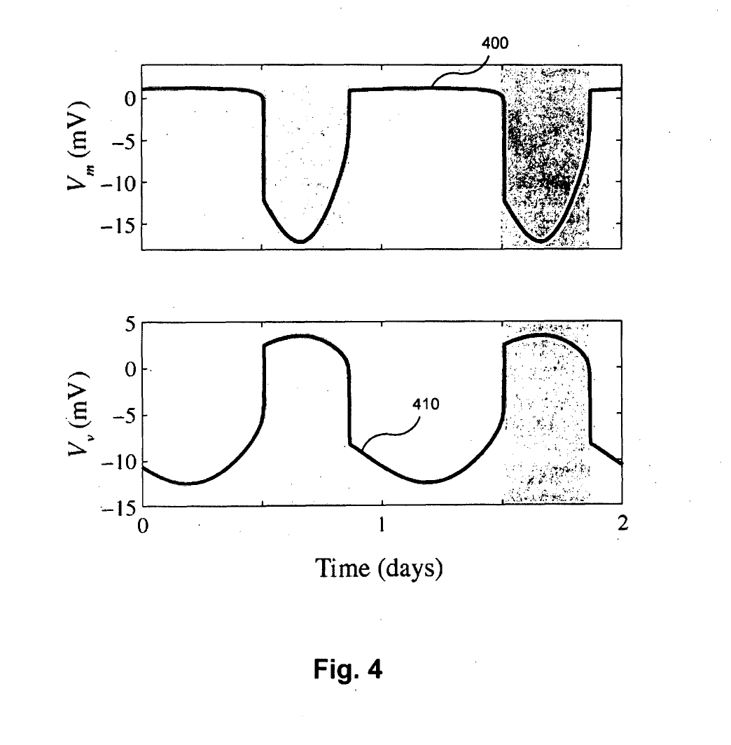

FIG. 4 contains plots of time series for the MA potential Vₘ (400) and the VLPO potential Vᵥ (410) for the simulated sleep-wake cycle of FIG. 3. During periods of wake (unshaded), the MA nucleus is activated (Vₘ is high) and the VLPO nucleus is suppressed (Vᵥ is low). During periods of sleep (shaded), the converse occurs: the MA nucleus is suppressed (Vₘ is low) and the VLPO nucleus is activated (Vᵥ is high), consistent with the physiology described above.

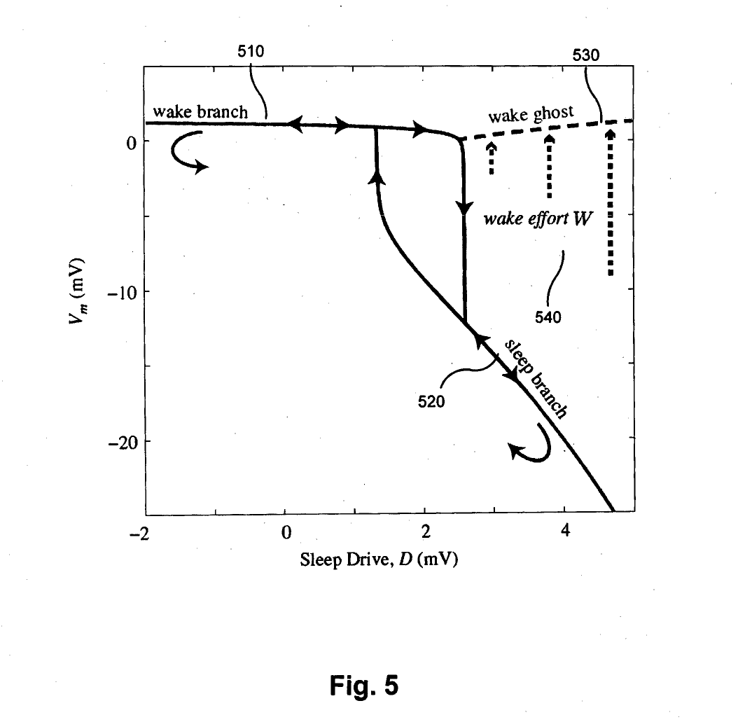

In FIG. 5, the MA potential 400 is plotted against the total sleep drive D 320 for the simulated sleep-wake cycle of FIG. 3. FIG. 5 illustrates an important feature of the PR model, namely the presence of hysteresis between the sleep and wake states. As D passes the low point in its oscillation, the model settles on the stable “wake branch” 510 with a high, almost constant value of Vₘ. As D increases above a threshold, the model leaves the “wake branch” and rapidly moves to the “sleep branch” 520 where it settles on low values of Vₘ. Between the two states are sudden transitions. However, as in a classic hysteresis, the wake-to-sleep transition occurs at a higher value of D (about 2.5 mV) than the D value (about 1.5 mV) at which sleep-to-wake transition does. Above the upper threshold, only the sleep state exists, so a short stimulus that artificially increases Vₘ will not cause prolonged waking. Between these two thresholds, two stable states exist, so a strong stimulus can push Vₘ to the wake branch 510, so that the person remains awake without continuing stimulus. The model behaviour is analogous to that of a flip-flop (bistable multivibrator) in electronics or a transformer in electromagnetics.

At high D (>2.5 mV) when the stable wake branch no longer exists, a near-stable “wake ghost” state exists, plotted with a dashed gray line 530 in FIG. 5. As shown in FIG. 5, the wake ghost trails on following the loss of the stable wake state. Ordinarily, as D increases past the normal wake-sleep transition (at D=2.5 mV), the system drops from the stable wake branch to the stable sleep branch. However, an additional “wake-effort” drive, denoted by W, can be applied to instead hold the system in the near-stable wake ghost, keeping it awake. Because of the wake ghost's remnant stability, at first only a small additional drive W is required to maintain waking in this way. Thus, wakefulness is maintained not by directly counteracting the growing ‘horizontal’ sleep drive D, but by compensating for it through the exertion of ‘vertical’ (in these coordinates) wake effort W. In other words, the compensatory W drive ‘pushes upwards’ on the system, keeping it in a high Vₘ state and preventing it from ‘dropping down’ to the stable sleep branch 520. As D increases, the wake ghost becomes less stable, and the amount of wake effort W required to remain in the wake state increases—the representative arrows 540 in FIG. 5 illustrate this trend.

It is apparent from FIG. 4 that the value Vₘ of the MA potential obtained by integrating the PR model is a useful proxy for the arousal state of an individual. However, Vₘ and the other state variables of the PR model cannot in general be measured non-invasively. What is therefore needed for a practical arousal state estimation system is a way of estimating the values of the state variables using physiological and behavioural variables relevant to the arousal state that can be measured non-invasively. Two such variables are core body temperature and actigraphy. Core body temperature is a physiological variable that exhibits a reliable diurnal rhythm (rising during wake, falling during sleep). It varies by approximately 1° C. across the day, usually reaching its peak in the late afternoon and its minimum in the early morning. Core body temperature is widely used in sleep research as a surrogate measure for the subject's circadian rhythm, which is closely related to the total sleep drive D in the PR model. Therefore, core body temperature readings may be used as a measurement for estimating arousal state based on the PR model.

An actigraphy signal is a behavioural variable representing the amount of bodily movement, measured using an actigraph, typically worn around the wrist, that uses an accelerometer to measure a person's movement. Typically, actigraphy data provides high readings when a subject is awake and low readings when the subject is asleep.

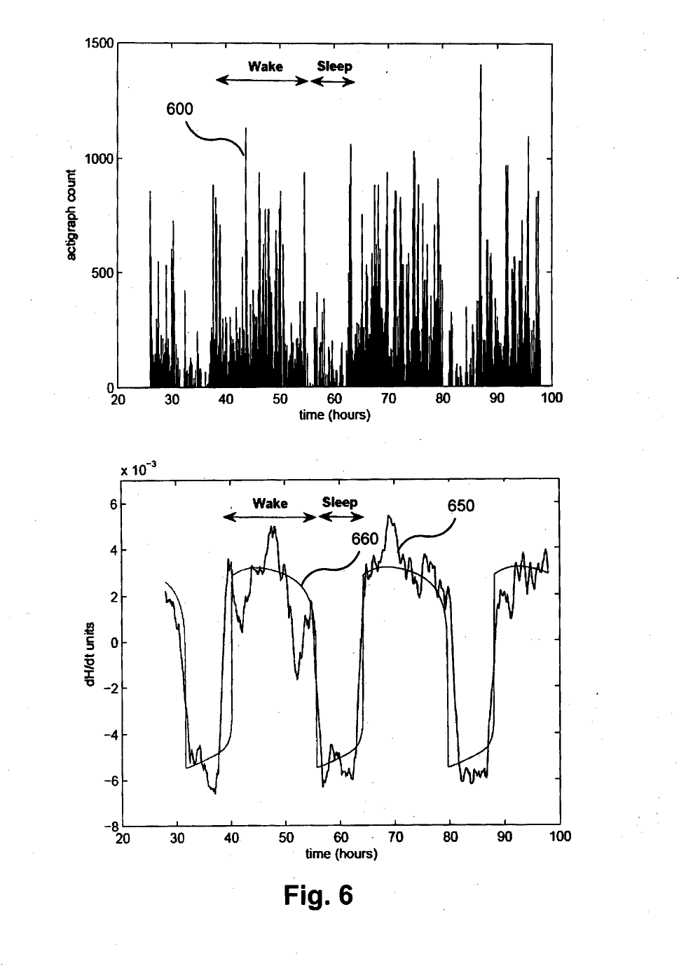

A typical example of raw actigraphy data, sampled every 30 seconds for 72 hours, is shown in the upper frame of FIG. 6. The raw actigraphy time series 600, despite being quite variable, does indeed display a diurnal structure—being on average higher during wake and lower during sleep. However, for the actigraphy data to be compatible with the PR model it needs to be preprocessed into a more suitable form. The preprocessing comprises four steps:

1. An arbitrary small number (0.01 in one implementation) is added to the raw actigraphy data to ensure the values remain strictly positive. The actigraphy data is then converted into logarithmic scale (base 10).
2. A moving average of the previous 2 hours of data (equivalent to 240 data points) is computed every 5 minutes.
3. The mean of the moving average time series is set to zero.
4. The zero-mean time series is then multiplied by a constant (0.03) to rescale the time series to match the values of dH/dt obtained from the PR model.

The lower frame of FIG. 6 is a plot of the preprocessed version 650 of the raw actigraphy time series 600, superimposed on the time derivative 660 of the homeostatic sleep drive H from the simulated sleep-wake cycle of FIG. 3. The preprocessed actigraphy time series 650 (denoted as B) corresponds closely enough to the time derivative 660 of the homeostatic sleep drive H for the preprocessed actigraphy time series 650 to be useful as a measurement for estimating the arousal state based on the PR model.

Other arousal-relevant physiological or behavioural measurements that could be used to estimate the state variable values of the PR model include: levels of natural or introduced chemicals in the body such as melatonin, stimulants, and sedatives; cardiovascular and respiratory measurements such as pulse and respiration rate; and electroencephalographic variables. Such physiological measurements could be made by analyzing a sample of the subject's blood, sweat, or saliva.

Kalman filtering (KF) is a widely used technique for recursively estimating the state of a dynamic system based on noisy observations (measurements) of the system. The state variables are modelled as Gaussian random variables, and their estimates are optimal in the sense that they minimize the variance of the error between the estimated and true state variable values. While many variants of the Kalman filter have been developed, they share several common features:

1. A mathematical model describing the dynamics of the system (states) being estimated in terms of first-order differential equations in the state variables with one or more parameters.
2. Observations (measurements) of the system, which are related to the states being estimated via a smooth “measurement function”.
3. Two noise parameters, both with a zero-mean Gaussian distribution, known as the process and measurement noise. The process noise models random inputs into the dynamic model, as well as compensating for any modelling errors and/or unmodelled dynamics by injecting uncertainty into the state covariance matrix. The effect of this is to make the Kalman filter place greater weight on the measurements relative to the model predictions. The measurement noise models any noise in the measurement process (e.g. from the sensor(s) being used), as well as compensating for any errors in the observation model by injecting uncertainty into the measurement covariance matrix.

All KF variants operate by performing a series of mathematical operations each time a new measurement (or set of measurements) becomes available. Broadly speaking these operations fall under two headings, namely the time-update (prediction) phase and the measurement-update (correction) phase, which are performed sequentially at each time instant. In the prediction phase, the vector of state variables at the current time instant is predicted from its value at the previous time instant. In the correction phase, the predicted state vector is corrected using the measurement vector at the current time instant.

For the disclosed system, the Unscented Kalman Filter (UKF) is employed. The UKF has two important advantages over the alternative Extended Kalman Filter: (i) its ability to more reliably handle highly nonlinear dynamic models such as the PR model, and (ii) its ability to provide more accurate estimates of unknown model parameters while simultaneously estimating the model state variables.

The UKF is formulated as follows. The nonlinear deterministic model of the evolution of the state vector x (of length Dₓ) over time t is a system of first-order differential equations with D_{λ} parameters:

$$\frac{d x}{d t} = f ( x ( t ) , u ( t ) , λ ) \qquad ( 20 )$$

where f is a Dₓ-vector of scalar functions, u(t) is a Dᵤ-vector of inputs, and λ is a D_{λ}-vector of system parameters.

The measurement vector y (of length D_{y}) is a function of the state vector x, with additive measurement noise:

y(t)=H(x(t),u(t),λ)+nₜ (21)

where H is the measurement function (actually a D_{y}-vector of scalar functions) and ηₜ is a D_{y}-vector of measurement noise.

The continuous-time model (20) may be integrated between time instants t and t+Δt to form the discrete-time nonlinear model of the evolution of the state vector x between time instants t and t+Δt as

x_{t+Δt}=F(xₜ,uₜ,λ)+εₜ (22)

where F is the system function (actually a Dₓ-vector of scalar functions) given by

$$F ( {x}_{t} , {u}_{t} , λ ) = {x}_{t} + {\int }_{t}^{t + Δ t} f ( x ( {t}^{′} ) , u ( {t}^{′} ) , λ ) d {t}^{′} \qquad ( 23 )$$

and εₜ is a Dₓ-vector of additive process noise. The system function F may be obtained from equation (23) using any numerical integration method, for example the fourth order Runge-Kutta method.

In the case of the PR model, the four state variables are Vᵥ, Vₘ, H, and C (so Dₓ=4), the parameters are as shown in Table 1 (so D_{λ}=20), the measurements are actigraphy (preprocessed) and core body temperature (so D_{y}=2), and the input vector u(t) is a delta function representing a discrete dose of caffeine at a single time instant (so Dᵤ=1). The system function F and the measurement function H are obtainable from the PR model equations (1) to (10).

The aim of any Kalman filter is to estimate the state vector x_{t+Δt} at the current time instant t+Δt given all the previous state estimates xₜ, x_{t-Δt}, . . . and the current and all previous measurement vectors y_{t+Δt}, yₜ, y_{t-Δt}, . . . . This estimate of x_{t+Δt} is the a posteriori, or complete, estimate which utilises all available information up to and including the current time instant, and is denoted as \hat{x}_{t+Δt|t+Δt}, or hereafter (to save subscripts) simply as \hat{x}. To compute \hat{x}, the UKF first computes the a priori estimate, or prediction, of the state vector at t+Δt utilising all the previous state estimates \hat{x}_{t|t}, \hat{x}_{t-Δt|t-Δt}, . . . and all the previous measurement vectors yₜ, y_{t-Δt}, . . . , but not the current measurement y_{t+Δt}, yielding a value denoted as \tilde{x}_{t+Δt|t}, or hereafter (to save subscripts) simply as \tilde{x}, by applying the model (22) as follows:

\tilde{x}=F(\hat{x}_{t|t},uₜ,λ) (24)

Next, the UKF computes the predicted measurement vector at the current time instant, using the current state prediction \tilde{x}, yielding a measurement prediction denoted as \tilde{y}_{t+Δt|t}, or hereafter (to save subscripts) simply as \tilde{y}, as follows:

\tilde{y}=H(\tilde{x},uₜ,λ) (25)

The UKF then forms the state estimate \hat{x} by correcting the prediction \tilde{x} by an amount proportional to the difference between the current measurement y_{t+Δt} and the measurement prediction \tilde{y}:

\hat{x}=\tilde{x}+K(y_{t+Δt}−\tilde{y}) (26)

The Dₓ-by-D_{y} Kalman gain matrix K is selected so that \hat{x} minimises the variance of the error between the estimated and true state variable values. The matrix K is given by

K=\tilde{P}_{xy}\tilde{P}_{yy}⁻¹ (27)

where \tilde{P}_{xy} is the (Dₓ-by-D_{y}) predicted cross-covariance matrix of the state variables and the measurement variables, and \tilde{P}_{yy} is the (D_{y}-by-D_{y}) predicted covariance matrix of the measurement variables.

The last step of the UKF is to estimate the covariance matrix (uncertainty) \hat{P}ₓₓ of the state estimate \hat{x}, as follows:

\hat{P}ₓₓ=\tilde{P}ₓₓ−K\tilde{P}_{xy}K^{T} (28)

where \tilde{P}ₓₓ is the (Dₓ-by-Dₓ) predicted covariance matrix of the state variables.

In the special case where the system is linear, the functions F and H may be replaced by system matrices F and H, and the Kalman filter equations outlined above can be implemented as a series of matrix operations. However, approximations are required to implement the Kalman filter methodology for nonlinear systems. The UKF uses the unscented transform (UT) to handle nonlinear process and observation models. The UT is a method for calculating the statistics of a Gaussian random variable that undergoes a nonlinear transformation. The UT computes a minimal set of deterministically-selected points, known as sigma points, that completely capture the true mean and covariance of the random variable. When propagated through the nonlinear function, a weighted sample mean and covariance of the sigma points is computed that is accurate to the second order.

The first step of the UT is the initialisation of 2Dₓ+1 sigma points χᵢ, i=0, . . . , 2Dₓ, for the state estimate from time t. The sigma points χᵢ are initialised using the mean x̅ and covariance P of the state variables as follows:

χ₀= x̅ (29)

χᵢ= x̅+(\sqrt{(Dₓ+λ)P})ᵢ, i=1, . . . ,Dₓ (30)

χ_{i+D}_{ₓ}= x̅−(\sqrt{(Dₓ+λ)P})ᵢ, i=1, . . . ,Dₓ (31)

where the square root is any matrix square root of choice, the subscript i denotes the i-th column of the matrix square root and the sigma point matrix, and λ=α²(Dₓ+κ)−Dₓ is a scaling parameter. The constants α and κ for the scaling parameter λ are given standard values with α=1 and κ=0.

To propagate the sigma points χ_{i,t} at time t to sigma points χ_{i,t+Δt} at time t+Δt, and subsequently to compute the corresponding “measurement” sigma points γ_{i,t+Δt}, the model equations (22) and (21) are applied:

χ_{i,t+Δt}=F(χ_{i,t},uₜ,λ) (32)

γ_{i,t+Δt}=H(χ_{i,t+Δt},uₜ,λ) (32)

The sigma points χ_{i,t+Δt} and the corresponding measurement sigma points γ_{i,t+Δt} are then used to compute several quantities required by the Kalman filter correction-phase equations (26) to (28): the state prediction \tilde{x}, the measurement prediction \tilde{y}, and the predicted covariances \tilde{P}ₓₓ, \tilde{P}_{xy}, and \tilde{P}_{yy}.

$$\begin{matrix}\tilde{x} = {\sum }_{i = 0}^{2 {D}_{x}} {W}_{i}^{( m )} {χ}_{i , t + Δ t} & ( 34 ) \\ \tilde{y} = {\sum }_{i = 0}^{2 {D}_{x}} {W}_{i}^{( m )} {γ}_{i , t + Δ t} & ( 35 ) \\ {\tilde{P}}_{xx} = {\sum }_{i = 0}^{2 {D}_{x}} {W}_{i}^{( c )} ( {χ}_{i , y + Δ t} - \tilde{x} ) {( {χ}_{i , y + Δ t} - \tilde{x} )}^{T} + {Q}_{t + Δ t} & ( 36 ) \\ {\tilde{P}}_{xy} = {\sum }_{i = 0}^{2 {D}_{x}} {W}_{i}^{( c )} ( {χ}_{i , y + Δ t} - \tilde{x} ) {( {γ}_{i , y + Δ t} - \tilde{y} )}^{T} & ( 37 ) \\ {\tilde{P}}_{yy} = {\sum }_{i = 0}^{2 {D}_{x}} {W}_{i}^{( c )} ( {γ}_{i , y + Δ t} - \tilde{y} ) {( {γ}_{i , y + Δ t} - \tilde{y} )}^{T} + {R}_{t + Δ t} & ( 38 )\end{matrix}$$

where Q_{t+Δt} and R_{t+Δt} are the covariances of the process noise ε and measurement noise η in the equations (22) and (21) at time t+Δt. The weights Wᵢ, whose superscript indicates either mean (m) or covariance (c), are given by

$$\begin{matrix}{W}_{0}^{( m )} = \frac{λ}{{D}_{x} + λ} & ( 39 ) \\ {W}_{0}^{( c )} = \frac{λ}{{D}_{x} + λ} + 1 - {α}^{2} + β & ( 40 ) \\ {W}_{i}^{( m )} = {W}_{i}^{( c )} = \frac{1}{2 ( {D}_{x} + λ )} , i = 1 , … , 2 {D}_{x} & ( 41 )\end{matrix}$$

The constant β is given a standard value of 2.

To estimate one or more of the parameters λ simultaneously with the state vector x, the augmented state vector approach is used. Under this approach, the model equations (20) and (21) are rewritten in terms of the augmented state vector x′, consisting of the original state vector x concatenated with the parameters to be estimated, which are assumed constant over time. The remaining parameters, with predetermined values, are placed in the parameter vector λ. The UKF may then be reformulated in terms of the augmented state vector x′. If the dynamics of the system are indeed stationary, the parameter estimates should converge to the true value, while the covariance of the parameter estimates should decrease steadily over time.

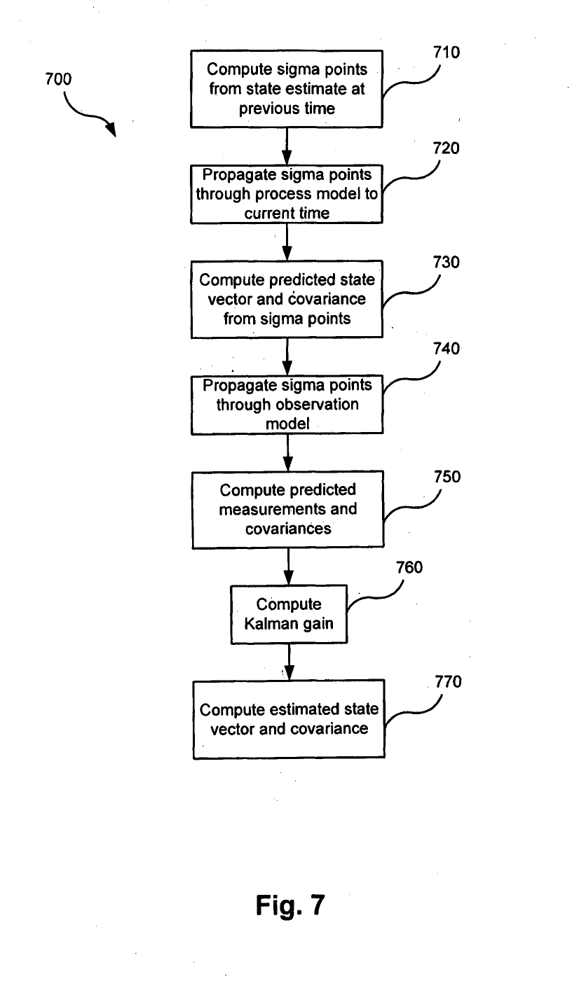

FIG. 7 is a flow diagram of a method 700 of estimating the arousal state and PR model parameters of a particular person at that instant by updating the PR model to the current time instant. The input to the method 700 at the current time instant t+Δt is a measurement vector y_{t+Δt} comprising the core body temperature T and actigraphy B of the person, and, if present, an input vector u(t). The augmented state vector x′ comprises the MA soma potential Vₘ, the VLPO soma potential Vᵥ, the homeostatic sleep drive H, the circadian sleep drive C, and some subset of the twenty PR model parameters listed in Table 1. The remaining parameters, whose values are predetermined and set to the values listed in Table 1, are placed in the fixed parameter vector λ. The output of the method 700 is the estimated augmented state vector \hat{x}′, which contains the state estimate \hat{x} and the parameter estimates \hat{λ}, and the augmented state covariance estimate \hat{P}_{x′x′}, which contains the estimated state covariance matrix \hat{P}ₓₓ and the estimated parameter covariance matrix \hat{P}_{λλ}. The method 700 uses the UKF to update the PR model to the current time instant.

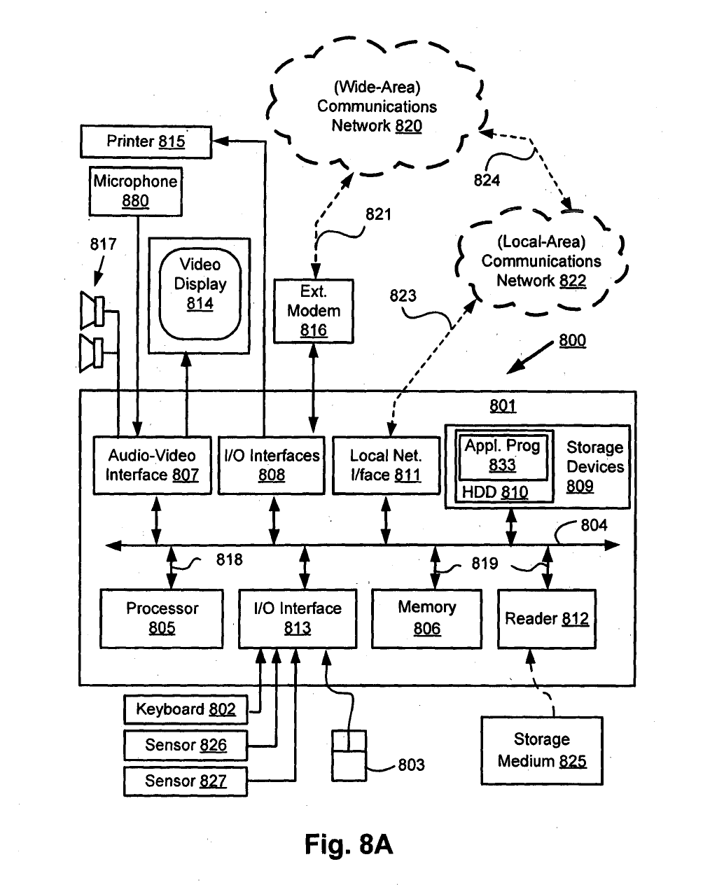

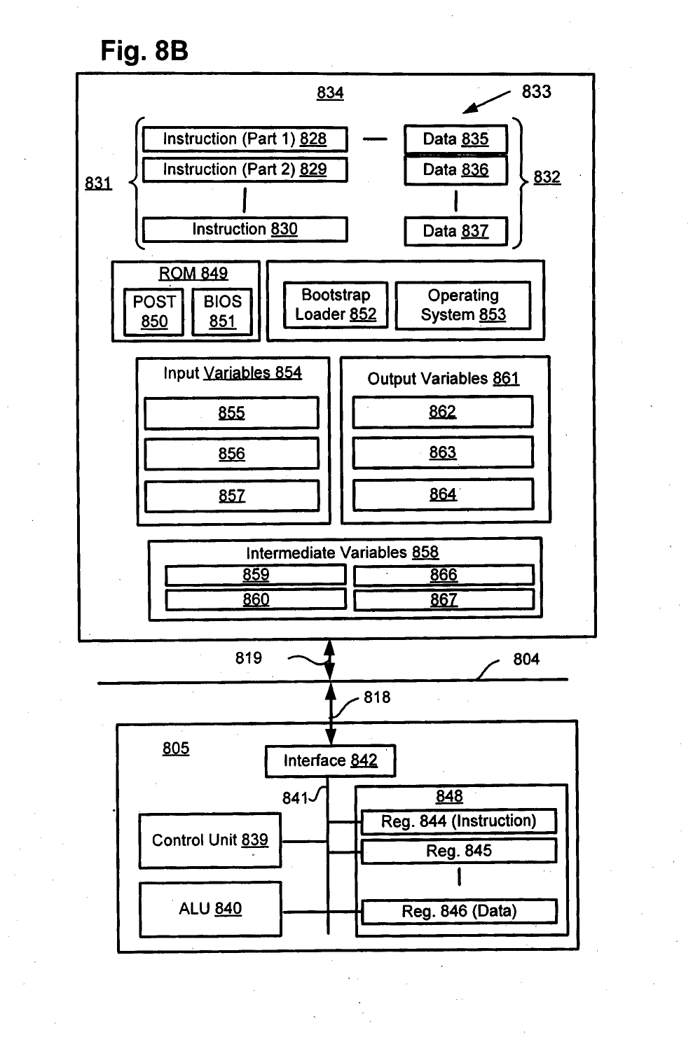

FIGS. 8A and 8B collectively form a schematic block diagram of a general purpose computer system 800, upon which the arousal state estimation method 700 described with reference to FIG. 7 can be practised. As seen in FIG. 8A, the computer system 800 is formed by a computer module 801, input devices such as a keyboard 802, a mouse pointer device 803, sensors 826 and 827, and a microphone 880, and output devices including a printer 815, a display device 814 and loudspeakers 817. An external Modulator-Demodulator (Modem) transceiver device 816 may be used by the computer module 801 for communicating to and from a communications network 820 via a connection 821. The network 820 may be a wide-area network (WAN), such as the Internet or a private WAN. Where the connection 821 is a telephone line, the modem 816 may be a traditional “dial-up” modem. Alternatively, where the connection 821 is a high capacity (eg: cable) connection, the modem 816 may be a broadband modem. A wireless modem may also be used for wireless connection to the network 820.

The sensors 826 and 827 may be a thermometer and an actigraph, configured to measure the core body temperature and actigraphy respectively of the person whose arousal state is to be estimated. The sensors 826 and 827 generate raw data values at time instants t separated by a predetermined time step Δt. Alternatively, the sensors 826 and 827 may be configured to measure other arousal-relevant physiological or behavioural variables as mentioned above.

The computer module 801 typically includes at least one processor unit 805, and a memory unit 806 for example formed from semiconductor random access memory (RAM) and semiconductor read only memory (ROM). The module 801 also includes an number of input/output (I/O) interfaces including an audio-video interface 807 that couples to the video display 814, loudspeakers 817 and microphone 880, an I/O interface 813 for the keyboard 802, mouse 803, sensors 826 and 827, and an interface 808 for the external modem 816 and printer 815. In some implementations, the modem 816 may be incorporated within the computer module 801, for example within the interface 808. The computer module 801 also has a local network interface 811 which, via a connection 823, permits coupling of the computer system 800 to a local computer network 822, known as a Local Area Network (LAN). As also illustrated, the local network 822 may also couple to the wide network 820 via a connection 824, which would typically include a so-called “firewall” device or device of similar functionality. The interface 811 may be formed by an Ethernet™ circuit card, a Bluetooth™ wireless arrangement or an IEEE 802.11 wireless arrangement.

The interfaces 808 and 813 may afford either or both of serial and parallel connectivity, the former typically being implemented according to the Universal Serial Bus (USB) standards and having corresponding USB connectors (not illustrated). Storage devices 809 are provided and typically include a hard disk drive (HDD) 810. Other storage devices such as a floppy disk drive and a magnetic tape drive (not illustrated) may also be used. A reader 812 is typically provided to interface with an external non-volatile source of data. A portable computer readable storage device 825, such as optical disks (e.g. CD-ROM, DVD), USB-RAM, and floppy disks for example may then be used as appropriate sources of data to the system 800.

The components 805 to 813 of the computer module 801 typically communicate via an interconnected bus 804 and in a manner which results in a conventional mode of operation of the computer system 800 known to those in the relevant art. Examples of computers on which the described arrangements can be practised include IBM-PC's and compatibles, Apple Mac™, or computer systems evolved therefrom.

The method 700 described hereinafter may be implemented as one or more software application programs 833 executable within the computer system 800. In particular, with reference to FIG. 8B, the steps of the method 700 are effected by instructions 831 in the software 833 that are carried out within the computer system 800. The software instructions 831 may be formed as one or more code modules, each for performing one or more particular tasks. The software 833 may also be divided into two separate parts, in which a first part and the corresponding code modules performs the method 700 and a second part and the corresponding code modules manage a user interface between the first part and the user.

The software 833 is generally loaded into the computer system 800 from a computer readable medium, and is then typically stored in the HDD 810, as illustrated in FIG. 8A, or the memory 806, after which the software 833 can be executed by the computer system 800. In some instances, the application programs 833 may be supplied to the user encoded on one or more storage media 825 and read via the corresponding reader 812 prior to storage in the memory 810 or 806. Computer readable storage media refers to any non-transitory tangible storage medium that participates in providing instructions and/or data to the computer system 800 for execution and/or processing. Examples of such storage media include floppy disks, magnetic tape, CD-ROM, DVD, a hard disk drive, a ROM or integrated circuit, USB memory, a magneto-optical disk, semiconductor memory, or a computer readable card such as a PCMCIA card and the like, whether or not such devices are internal or external to the computer module 801. A computer readable storage medium having such software or computer program recorded on it is a computer program product. The use of such a computer program product in the computer module 801 effects an apparatus for estimating the arousal state of a person.

Alternatively, the software 833 may be read by the computer system 800 from the networks 820 or 822 or loaded into the computer system 800 from other computer readable media. Examples of transitory or non-tangible computer readable transmission media that may also participate in the provision of software, application programs, instructions and/or data to the computer module 801 include radio or infra-red transmission channels as well as a network connection to another computer or networked device, and the Internet or Intranets including e-mail transmissions and information recorded on Websites and the like.

The second part of the application programs 833 and the corresponding code modules mentioned above may be executed to implement one or more graphical user interfaces (GUIs) to be rendered or otherwise represented upon the display 814. Through manipulation of typically the keyboard 802 and the mouse 803, a user of the computer system 800 and the application may manipulate the interface in a functionally adaptable manner to provide controlling commands and/or input to the applications associated with the GUI(s). Other forms of functionally adaptable user interfaces may also be implemented, such as an audio interface utilizing speech prompts output via the loudspeakers 817 and user voice commands input via the microphone 880.

FIG. 8B is a detailed schematic block diagram of the processor 805 and a “memory” 834. The memory 834 represents a logical aggregation of all the memory devices (including the HDD 810 and semiconductor memory 806) that can be accessed by the computer module 801 in FIG. 8A.

When the computer module 801 is initially powered up, a power-on self-test (POST) program 850 executes. The POST program 850 is typically stored in a ROM 849 of the semiconductor memory 806. A program permanently stored in a hardware device such as the ROM 849 is sometimes referred to as firmware. The POST program 850 examines hardware within the computer module 801 to ensure proper functioning, and typically checks the processor 805, the memory (809, 806), and a basic input-output systems software (BIOS) module 851, also typically stored in the ROM 849, for correct operation. Once the POST program 850 has run successfully, the BIOS 851 activates the hard disk drive 810. Activation of the hard disk drive 810 causes a bootstrap loader program 852 that is resident on the hard disk drive 810 to execute via the processor 805. This loads an operating system 853 into the RAM memory 806 upon which the operating system 853 commences operation. The operating system 853 is a system level application, executable by the processor 805, to fulfil various high level functions, including processor management, memory management, device management, storage management, software application interface, and generic user interface.

The operating system 853 manages the memory (809, 806) in order to ensure that each process or application running on the computer module 801 has sufficient memory in which to execute without colliding with memory allocated to another process. Furthermore, the different types of memory available in the system 800 must be used properly so that each process can run effectively. Accordingly, the aggregated memory 834 is not intended to illustrate how particular segments of memory are allocated (unless otherwise stated), but rather to provide a general view of the memory accessible by the computer system 800 and how such is used.

The processor 805 includes a number of functional modules including a control unit 839, an arithmetic logic unit (ALU) 840, and a local or internal memory 848, sometimes called a cache memory. The cache memory 848 typically includes a number of storage registers 844-846 in a register section. One or more internal buses 841 functionally interconnect these functional modules. The processor 805 typically also has one or more interfaces 842 for communicating with external devices via the system bus 804, using a connection 818.

The application program 833 includes a sequence of instructions 831 that may include conditional branch and loop instructions. The program 833 may also include data 832 which is used in execution of the program 833. The instructions 831 and the data 832 are stored in memory locations 828-830 and 835-837 respectively. Depending upon the relative size of the instructions 831 and the memory locations 828-830, a particular instruction may be stored in a single memory location as depicted by the instruction shown in the memory location 830. Alternately, an instruction may be segmented into a number of parts each of which is stored in a separate memory location, as depicted by the instruction segments shown in the memory locations 828-829.

In general, the processor 805 is given a set of instructions, for example including the method 700, which are executed therein. The processor 805 then waits for a subsequent input, to which it reacts to by executing another set of instructions. Each input may be provided from one or more of a number of sources, including data generated by one or more of the input devices 802, 803, data received from an external source across one of the networks 820, 822, data retrieved from one of the storage devices 806, 809 or data retrieved from a storage medium 825 inserted into the corresponding reader 812. The execution of a set of the instructions may in some cases result in output of data. Execution may also involve storing data or variables to the memory 834.

The disclosed methods use input variables 854, that are stored in the memory 834 in corresponding memory locations 855-858. The disclosed methods produce output variables 861, that are stored in the memory 834 in corresponding memory locations 862-865. Intermediate variables may be stored in memory locations 859, 860, 866 and 867.

The register section 844-846, the arithmetic logic unit (ALU) 840, and the control unit 839 of the processor 805 work together to perform sequences of micro-operations needed to perform “fetch, decode, and execute” cycles for every instruction in the instruction set making up the program 833. Each fetch, decode, and execute cycle comprises:

(a) a fetch operation, which fetches or reads an instruction 831 from a memory location 828;
(b) a decode operation in which the control unit 839 determines which instruction has been fetched; and
(c) an execute operation in which the control unit 839 and/or the ALU 840 execute the instruction.

Thereafter, a further fetch, decode, and execute cycle for the next instruction may be executed. Similarly, a store cycle may be performed by which the control unit 839 stores or writes a value to a memory location 832.

Each step or sub-process in the method 700 is associated with one or more segments of the program 833, and is performed by the register section 844-847, the ALU 840, and the control unit 839 in the processor 805 working together to perform the fetch, decode, and execute cycles for every instruction in the instruction set for the noted segments of the program 833.

The method 700 may alternatively be implemented in dedicated hardware such as one or more integrated circuits performing the functions or sub functions of the method 700. Such dedicated hardware may include graphic processors, digital signal processors, or one or more microprocessors and associated memories.

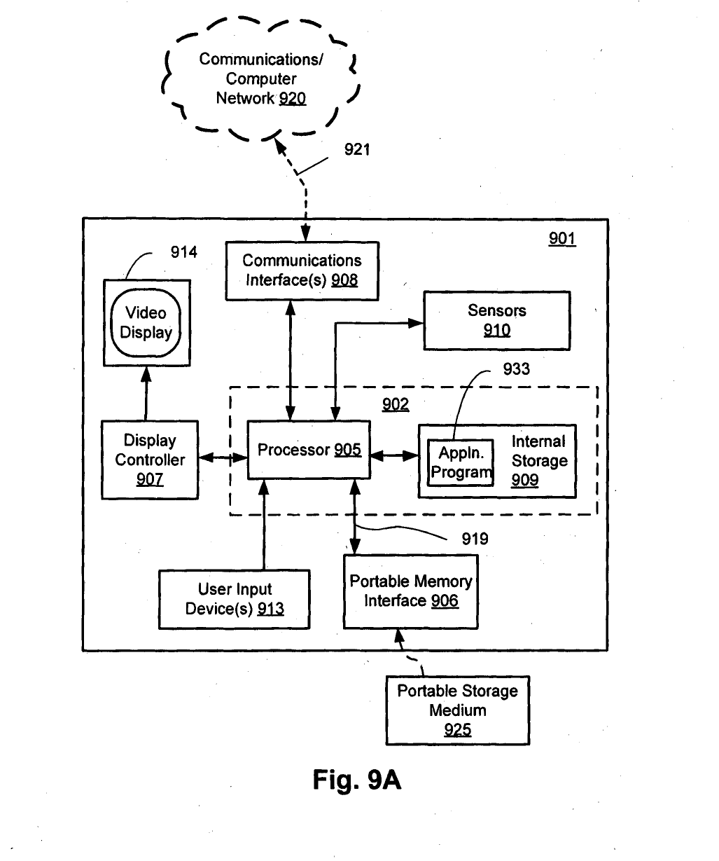

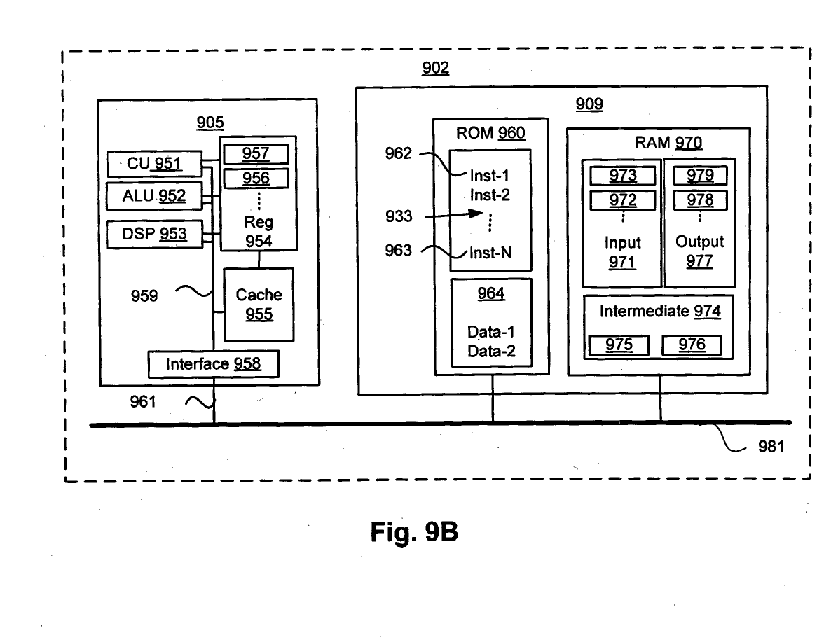

FIGS. 9A and 9B collectively form a schematic block diagram of a general purpose electronic device 901 including embedded components, upon which the method 700 may alternatively be implemented.

As seen in FIG. 9A, the electronic device 901 comprises an embedded controller 902. Accordingly, the electronic device 901 may be referred to as an “embedded device.” In the present example, the controller 902 has a processing unit (or processor) 905 which is bi-directionally coupled to an internal storage module 909. The storage module 909 may be formed from non-volatile semiconductor read only memory (ROM) 960 and semiconductor random access memory (RAM) 970, as seen in FIG. 9B. The RAM 970 may be volatile, non-volatile or a combination of volatile and non-volatile memory.

The electronic device 901 includes a display controller 907, which is connected to a video display 914, such as a liquid crystal display (LCD) panel or the like. The display controller 907 is configured for displaying graphical images on the video display 914 in accordance with instructions received from the embedded controller 902, to which the display controller 907 is connected.

The electronic device 901 also includes user input devices 913 which are typically formed by keys, a keypad, or like controls. In some implementations, the user input devices 913 may include a touch sensitive panel physically associated with the display 914 to collectively form a touch-screen. Such a touch-screen may thus operate as one form of graphical user interface (GUI) as opposed to a prompt or menu driven GUI typically used with keypad-display combinations. Other forms of user input devices may also be used, such as a microphone (not illustrated) for voice commands or a joystick/thumb wheel (not illustrated) for ease of navigation about menus.

As seen in FIG. 9A, the electronic device 901 also comprises a portable memory interface 906, which is coupled to the processor 905 via a connection 919. The portable memory interface 906 allows a complementary portable computer readable storage medium 925 to be coupled to the electronic device 901 to act as a source or destination of data or to supplement the internal storage module 909. Examples of such interfaces permit coupling with portable computer readable storage media such as Universal Serial Bus (USB) memory devices, Secure Digital (SD) cards, Personal Computer Memory Card International Association (PCMIA) cards, optical disks and magnetic disks.

The electronic device 901 also has a communications interface 908 to permit coupling of the electronic device 901 to a computer or communications network 920 via a connection 921. The connection 921 may be wired or wireless. For example, the connection 921 may be radio frequency or optical. An example of a wired connection includes Ethernet. Further, an example of wireless connection includes Bluetooth™ type local interconnection, Wi-Fi (including protocols based on the standards of the IEEE 802.11 family), Infrared Data Association (IrDa) and the like.

The electronic device 901 also includes sensors 910 that are configured to measure one or more properties of the person whose arousal state is to be estimated. For example, the sensors 910 may include a thermometer for monitoring core temperature and an actigraph for measuring activity. The sensors 910 are connected to the embedded controller 902 and provide information describing the measured properties in a format and according to a protocol compatible, with the embedded controller 902.

The method 700 may be implemented as one or more software application programs 933 executable within the embedded controller 902. In particular, with reference to FIG. 9B, the steps of the method 700 are effected by instructions in the software 933 that are carried out within the embedded controller 902. The software instructions may be formed as one or more code modules, each for performing one or more particular tasks. The software may also be divided into two separate parts, in which a first part and the corresponding code modules performs the method 700 and a second part and the corresponding code modules manage a user interface between the first part and the user.

The software 933 of the embedded controller 902 is typically stored in the non-volatile ROM 960 of the internal storage module 909. The software 933 stored in the ROM 960 can be updated when required from a computer readable medium. The software 933 can be loaded into and executed by the processor 905. In some instances, the processor 905 may execute software instructions that are located in RAM 970. Software instructions may be loaded into the RAM 970 by the processor 905 initiating a copy of one or more code modules from ROM 960 into RAM 970. Alternatively, the software instructions of one or more code modules may be pre-installed in a non-volatile region of RAM 970 by a manufacturer. After one or more code modules have been located in RAM 970, the processor 905 may execute software instructions of the one or more code modules.

The application program 933 is typically pre-installed and stored in the ROM 960 by a manufacturer, prior to distribution of the electronic device 901. However, in some instances, the application programs 933 may be supplied to the user encoded on the computer readable storage medium 925 and read via the portable memory interface 906 of FIG. 9A prior to storage in the internal storage module 909. Computer readable storage media refers to any non-transitory tangible storage medium that participates in providing instructions and/or data to the embedded controller 902 for execution and/or processing. Examples of such storage media include floppy disks, magnetic tape, CD-ROM, DVD, a hard disk drive, a ROM or integrated circuit, USB memory, a magneto-optical disk, flash memory, or a computer readable card such as a PCMCIA card and the like, whether or not such devices are internal or external of the electronic device 901. A computer readable medium having such software or computer program recorded on it is a computer program product. The use of such a computer program product in the electronic device 901 effects an apparatus for estimating the arousal state of a person.

In another alternative, the software application program 933 may be read by the processor 905 from the network 920, or loaded into the embedded controller 902 from other computer readable media. Examples of transitory or non-tangible computer readable transmission media that may also participate in the provision of software, application programs, instructions and/or data to the electronic device 901 include radio or infra-red transmission channels as well as a network connection to another computer or networked device, and the Internet or Intranets including e-mail transmissions and information recorded on Websites and the like.

The second part of the application programs 933 and the corresponding code modules mentioned above may be executed to implement one or more graphical user interfaces (GUIs) to be rendered or otherwise represented upon the display 914 of FIG. 9A. Through manipulation of the user input device 913 (e.g., the keypad), a user of the electronic device 901 and the application programs 933 may manipulate the interface in a functionally adaptable manner to provide controlling commands and/or input to the applications associated with the GUI(s). Other forms of functionally adaptable user interfaces may also be implemented, such as an audio interface utilizing speech prompts output via loudspeakers (not illustrated) and user voice commands input via the microphone (not illustrated).

FIG. 9B illustrates in detail the embedded controller 902 having the processor 905 for executing the application programs 933 and the internal storage 909. The internal storage 909 comprises read only memory (ROM) 960 and random access memory (RAM) 970. The processor 905 is able to execute the application programs 933 stored in one or both of the connected memories 960 and 970. When the electronic device 901 is initially powered up, a system program resident in the ROM 960 is executed. The application program 933 permanently stored in the ROM 960 is sometimes referred to as “firmware”. Execution of the firmware by the processor 905 may fulfil various functions, including processor management, memory management, device management, storage management and user interface.

The processor 905 typically includes a number of functional modules including a control unit (CU) 951, an arithmetic logic unit (ALU) 952 and a local or internal memory comprising a set of registers 954 which typically contain atomic data elements 956, 957, along with internal buffer or cache memory 955. One or more internal buses 959 interconnect these functional modules. The processor 905 typically also has one or more interfaces 958 for communicating with external devices via system bus 981, using a connection 961.

The application program 933 includes a sequence of instructions 962 through 963 that may include conditional branch and loop instructions. The program 933 may also include data, which is used in execution of the program 933. This data may be stored as part of the instruction or in a separate location 964 within the ROM 960 or RAM 970.

In general, the processor 905 is given a set of instructions, for example including the method 700, which are executed therein. This set of instructions may be organised into blocks, which perform specific tasks or handle specific events that occur in the electronic device 901. Typically, the application program 933 waits for events and subsequently executes the block of code associated with that event. Events may be triggered in response to input from a user, via the user input devices 913 of FIG. 9A, as detected by the processor 905. Events may also be triggered in response to other sensors and interfaces in the electronic device 901.

The execution of a set of the instructions may require numeric variables to be read and modified. Such numeric variables are stored in the RAM 970. The disclosed method uses input variables 971 that are stored in known locations 972, 973 in the memory 970. The input variables 971 are processed to produce output variables 977 that are stored in known locations 978, 979 in the memory 970. Intermediate variables 974 may be stored in additional memory locations in locations 975, 976 of the memory 970. Alternatively, some intermediate variables may only exist in the registers 954 of the processor 905.

The execution of a sequence of instructions is achieved in the processor 905 by repeated application of a fetch-execute cycle. The control unit 951 of the processor 905 maintains a register called the program counter, which contains the address in ROM 960 or RAM 970 of the next instruction to be executed. At the start of the fetch execute cycle, the contents of the memory address indexed by the program counter is loaded into the control unit 951. The instruction thus loaded controls the subsequent operation of the processor 905, causing for example, data to be loaded from ROM memory 960 into processor registers 954, the contents of a register to be arithmetically combined with the contents of another register, the contents of a register to be written to the location stored in another register and so on. At the end of the fetch execute cycle the program counter is updated to point to the next instruction in the system program code. Depending on the instruction just executed this may involve incrementing the address contained in the program counter or loading the program counter with a new address in order to achieve a branch operation.

Each step or sub-process in the processes of the methods described below is associated with one or more segments of the application program 933, and is performed by repeated execution of a fetch-execute cycle in the processor 905 or similar programmatic operation of other independent processor blocks in the electronic device 901.

The method 700 starts at the step 710, where the processor 805 or 905 computes the sigma points χ_{i,t} from the state estimate at the previous time t using equations (29), (30), and (31). At the next step 720, the processor 805 or 905 takes the sigma points χ_{i,t} from time t and propagates them through the process model to produce sigma points χ_{i,t+Δt} at time t+Δt using equation (32). Step 730 follows, at which the processor 805 or 905 applies equation (34) and (36) to the sigma points χ_{i,t+Δt} to compute the predicted state vector \tilde{x} and the predicted state covariance matrix \tilde{P}ₓₓ respectively. Then at step 740, the processor 805 or 905 propagates the sigma points χ_{i,t+Δt} through the observation model using equation (33) to produce a set of measurement sigma points γ_{i,t+Δt}. At the next step 750, the processor 805 or 905 applies equations (35), (38) and (37) to the sigma points χ_{i,t+Δt} and γ_{i,t+Δt} to compute the predicted measurement vector \tilde{y}, the measurement covariance matrix \tilde{P}_{yy}, and the state/measurement cross-covariance matrix \tilde{P}_{xy}, respectively. Step 760 follows, at which the processor 805 or 905 computes the Kalman gain matrix K using equation (27). The processor 805 or 905 then at step 770 calculates the estimated state vector \hat{x} and the state covariance matrix estimate \hat{P}ₓₓ equations (26) and (28) respectively. Step 770 corrects the state prediction and its associated covariance by combining them with information contained in the measurements via the measurement, the measurement covariance, and the Kalman gain. The method 700 then concludes.

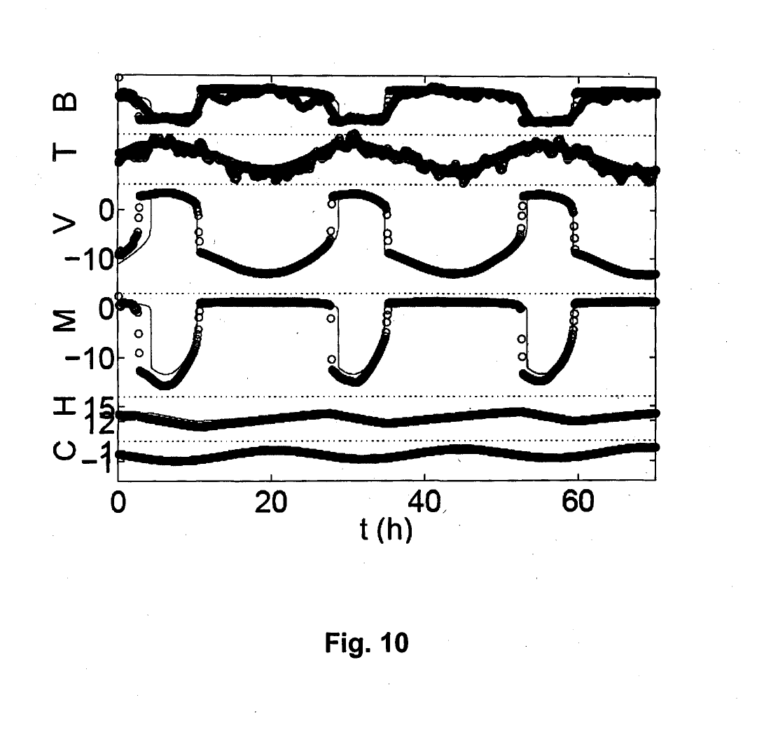

FIG. 10 contains plots of the inputs (measurements) to and outputs (state variable estimates) of the arousal state estimation method 700 described above with reference to FIG. 7. The arousal state estimation method 700 is run iteratively on a measurement dataset representing a 72-hour period. The top two time series in FIG. 10 show the components of the measurement data set: the preprocessed actigraphy time series 650 of FIG. 6, denoted as B, and a corresponding core body temperature time series, denoted as T (each shown as dark circles). The arousal state estimation method 700 is run each time new measurements become available, which is every 5 minutes for the results shown in FIG. 10. To ensure the accuracy of the predictions of the PR model, the numerical integration scheme uses a time step Δt of 6 seconds, which means the PR model must be integrated 50 times between successive sets of measurements.

The measurement noise covariance matrix R and process noise covariance matrix Q are assumed to be diagonal and constant over time. The measurement noise covariance values are calculated assuming a signal-to-noise ratio of 2.5, while the process noise covariance values are set to 10% of the initial state vector values.

The lower four time series in FIG. 10 are the resulting estimates of the four state variables Vᵥ, Vₘ, H, and C (denoted as V, M, H, and C respectively) of the PR model, each shown as circles, at each measurement time. The smooth line underlying each time series shows the actual evolution of the state variables, obtained by integrating the PR model directly, starting from the initial state variable values. The close correspondence between the estimated values and the actual values for each state variable shows that the arousal state estimation method 700 was able to reliably track the person's arousal state as modelled by the PR model.

The arousal state estimation method 700 can continue to run purely predictively, i.e. in the absence of measurements, to predict (for example) when a person will next fall asleep, or what a person's arousal state will be a given time in the future. To run predictively, the arousal state estimation method 700 performs steps 710, 720, and 730, and then skips the remaining steps. In predictive mode, the state covariance estimate \hat{P}ₓₓ will grow over time. This means that the state predictions become increasingly inaccurate until at some point they become useless, an interval known in the weather forecasting field as the “prediction horizon”. However, a reliable prediction horizon of several hours, or possibly even days, is not unreasonable to expect as proven in a number of test cases.

Techniques from control theory may be used to predict the arousal state according to the PR-model under various future input scenarios and thereby recommend an input scenario that will alter the future arousal state in a desired manner, either to hasten sleep or to delay it.

In an example of such an application, a subject's physiological and behavioural measurements are recorded over an extended time period at regular intervals on a wearable device such as a specially configured wristwatch. On arrival at their place of employment, these measurements may be wirelessly uploaded to a computer system or an embedded device and used as inputs to the arousal state estimation method 700, which is run repeatedly to estimate the subject's past arousal state and model parameters over the period of recording. Once the measurements are exhausted, the arousal state estimation method 700 is run repeatedly in predictive mode with the estimated model parameters to predict the subject's future arousal state in a variety of future input scenarios within the prediction horizon. The input scenario for which the predicted arousal state approximates a desired arousal state over the prediction period, e.g. “drink a cup of coffee in two hours”, is determined and recommended for administration to the subject. Further, the arousal state estimation method gives mathematically precise advice on which control inputs can be used to steer the system toward a desired state, and whether the desired state is reachable. The weights it assigns to various input streams can also be used to determine the relative usefulness of each measurement type.

As an alternative, the measurements may be uploaded using wired technology. Further, the measurements may be uploaded to a wearable or adjacent device continuously, rather than just at the start of a shift.

Determination of the physiological model parameters for an individual subject can facilitate diagnosis of disorders—abnormal values of a parameter may be found to be associated with a particular medical condition (e.g. obstructive sleep apnea, narcolepsy for example). Alternatively, conditions such as shiftwork-related sleep disorders or jetlag-related sleep disruption may be diagnosed in a similar manner. Such diagnoses could be used to guide counteractions, via administration of drugs, light, or behavioural changes.

The arrangements described are applicable for example to the medical, transport, manufacturing, and power industries.

The foregoing describes only some embodiments of the present invention, and modifications and/or changes can be made thereto without departing from the scope and spirit of the invention, the embodiments being illustrative and not restrictive.

1. A method of estimating an arousal state of a person, the method comprising:
providing one or more measurements of a physiological or behavioural variable relevant to the arousal state of the person; and
updating a nonlinear physiologically based time-dependent model of arousal using the one or more measurements of the physiological or behavioural variable, thereby estimating an augmented state vector of the model,
wherein the estimated augmented state vector of the model includes the arousal state of the person and one or more parameters of the model.

2. The method of claim 1, wherein the physiological or behavioural variable is core body temperature of the person.

3. The method of claim 1, wherein the physiological or behavioural variable is activity of the person.

4. The method of claim 3, further comprising preprocessing the measurement of activity.

5. The method of claim 1, wherein the updating uses an unscented Kalman filter.

6. The method of claim 1, wherein the updating uses one or more inputs to the model.

7. The method of claim 6, wherein the input is a stimulant or sedative dosage.

8. The method of claim 6, wherein the updating does not use any measurement, the method further comprising:
iterating the updating up to a future time instant over one or more scenarios of inputs to the model to obtain a predicted arousal state at the future time instant, and
determining an input scenario for which the predicted arousal state at the future time instant approximates a desired arousal state.

9. The method of claim 1, wherein the providing comprises retrieving the one or more measurements from a device wearable by the person.

10. The method of claim 9, wherein the one or more measurements are retrieved from a device wearable by the person.

11. A system for estimating an arousal state of a person, the system comprising:
one or more sensors configured to measure one or more measurements of a physiological or behavioural variable relevant to the arousal state of the person;
a memory, and
a processor configured to execute program instructions stored in the memory, the program instructions being configured to cause the processor to update a nonlinear physiologically based time-dependent model of arousal using the one or more measurements of the physiological or behavioural variable, thereby estimating an augmented state vector of the model, wherein the estimated augmented state vector of the model includes the arousal state of the person and one or more parameters of the model.

12. Computer program code configured to cause, when executed by a processor, the processor to carry out a method of estimating an arousal state of a person, the method comprising updating a nonlinear physiologically based time-dependent model of arousal using one or more measurements of a physiological or behavioural variable relevant to the arousal state of the person, thereby estimating an augmented state vector of the model, wherein the estimated augmented state vector of the model includes the arousal state of the person and one or more parameters of the model.

13. A system of claim 1, wherein the physiological or behavioural variable is core body temperature of the person.

14. The system of claim 1, wherein the physiological or behavioural variable is activity of the person.

15. The system of claim 14, wherein the program instructions are further configured to cause the processor to preprocess the measurement of activity.

16. The system of claim 1, wherein the program instructions are configured to cause the processor to update the nonlinear physiologically based time-dependent model of arousal using an unscented Kalman filter.

17. The system of claim 1, wherein the program instructions are configured to cause the processor to update the nonlinear physiologically based time-dependent model of arousal using one or more inputs to the model.

18. The system of claim 17, wherein the input is a stimulant or sedative dosage.

19. The system of claim 17, wherein the program instructions are configured to cause the processor to:
update the nonlinear physiologically based time-dependent model of arousal note using any measurement,
iterating the update up to a future time instant over one or more scenarios of inputs to the model to obtain a predicted arousal state at the future time instant, and
determine an input scenario for which the predicted arousal state at the future time instant approximates a desired arousal state.

20. The system of claim 1, wherein the one or more sensors are incorporated in a device wearable by the person.
